(19) United States
(12) Patent Application Publication
(10) Pub. No.: US 2016/0262654 A1
(43) Pub. Date: Sep. 15, 2016

(54) TRACKING SIGNALS FOR CATHETER

(71) Applicants: Steven R. Wedan, Savage, MN (US); Thomas W. Lloyd, Eagan, MN (US); Milton Noe Turcios, Farmington, MN (US); Daniel Nathan Sunnarborg, Bloomington, MN (US)

(72) Inventors: Steven R. Wedan, Savage, MN (US); Thomas W. Lloyd, Eagan, MN (US); Milton Noe Turcios, Farmington, MN (US); Daniel Nathan Sunnarborg, Bloomington, MN (US)

(21) Appl. No.: 14/642,049

(22) Filed: Mar. 9, 2015

(51) Int. Cl. A61B 5/06; A61B 5/055

(52) U.S. Cl. CPC A61B 5/062; A61B 5/055

(57) ABSTRACT

A method for projecting a broad tracking signal received by an inductively coupled element, such as a transformer during an MR tracking sequence is provided. By varying projection planes, the signal acquired from the transformer along the transmission line can be used to depict the body of the actively tracked medical device, such as the shaft or deflection region of a catheter. This may be achieved by interpolating a line between the position of the transformer element within the transmission line and the tracking coil. A curvature may be added to the line segment and gradually increased until the arc length of the line segment is approximately equal to the predefined length. The direction of the curve may be determined by virtually connecting the transformer position to the distal most tracking coil position, then the curve of the line segment is increased towards the proximal coil position.

### BACKGROUND OF THE INVENTION

The present invention relates generally to a method for projecting a broad tracking signal received by an inductively coupled element, such as a transformer during an MR tracking sequence.

### BRIEF SUMMARY OF THE INVENTION

Interventional medical procedures are typically performed using x-ray fluoroscopy imaging to guide the procedure. X-ray imaging is used to visualize devices and anatomy inside a patient. However, because of its superior soft tissue imaging capabilities, many procedures can benefit from the utilization of magnetic resonance imaging (MRI) for guidance rather than x-ray imaging.

In many MRI guided interventional procedures, it is advantageous to locate the device within the patient using active MR tracking. Active MR tracking is a well-known technique wherein one or more MR receive coils (“tracking coils”) are incorporated into a device, and tracking pulse sequences are used to locate the coils. Tracking sequences generally locate a tracking coil by finding their location in each of three orthogonal planes. Such planes correspond to an x-y-z coordinate system, but the relationship between this x-y-z coordinate system and the patient or MR system may be arbitrary.

Tracking coils must be connected to circuitry to receive the MR signals from the coils. This is often facilitated by a transmission line, such as a coaxial cable. However, if the length of the coaxial cable is sufficiently long, safety issues arise due to radio frequency (RF) coupling to the transmission line. Several techniques have been proposed to make transmission lines safe for use in MRI. One technique, for instance, incorporates miniature inductively coupled elements, such as transformers, along the transmission line to reduce the common mode RF currents.

### PROBLEM TO BE SOLVED

An inductively coupling element (ICE), such as a transformer, may pick up MR signals from surrounding tissues, and therefore behave as tracking coils themselves. Depending on the orientation of the ICE relative to the tracking sequence projections, the MR signal received from the ICE may have equal or greater signal intensity than the MR signal picked up from the MR tracking coil. If the unwanted signal from the ICE is larger than the signal received by the MR tracking coil, the tracking coil's location may be erroneously determined to be the ICE's location. This, in turn, results in an erroneous tracking location for the medical device. FIG. 2 shows ICE orientations that maximize and minimize signal pickup by the ICE.

In addition, even in the absence of any ICE, MR tracking coils may receive more or less signal, depending on their orientation to the tracking sequence projections.

Thus, what is needed is an improved method for projecting a broad tracking signal received by an inductively coupled element, such as a transformer during an MR tracking sequence.

### BRIEF DESCRIPTION OF THE DRAWINGS

For a better understanding of the invention, and to show how the same may be carried into effect, reference will now be made, by way of example, to the accompanying drawings, in which:

FIG. 1 depicts an example of an x-y-z coordinate system and an alternative orthogonal x′-y′-z′ coordinate system in dashed lines that is offset from the original.

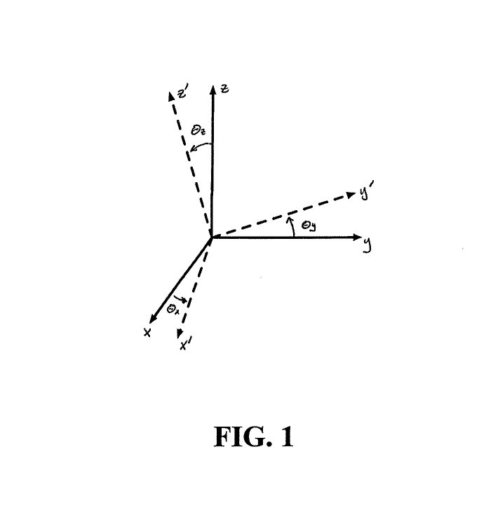

FIG. 2 illustrates three orientations for an ICE relative to the MR gradient along the projection axis used for tracking.

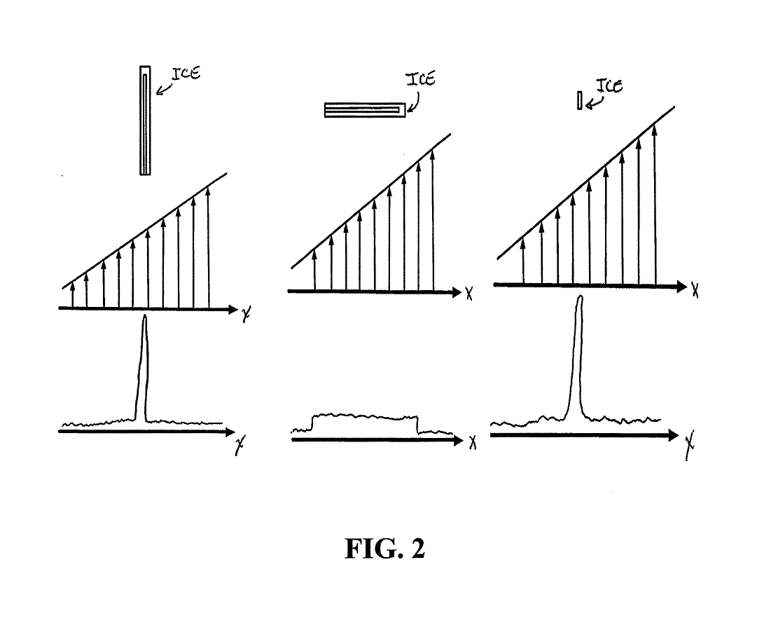

FIGS. 3 and 4 depict an ICE projecting a signal onto the z axis and showing that the z-axis is larger than the tracking coil signals projected onto the same axis.

FIG. 5A-5B depict the orientations of the ICE and tracking coil relative to the tracking project showing that the tracking coil signal is larger than the ICE signal, in accordance with the invention.

### DETAILED DESCRIPTION OF THE INVENTION

The MR signal intensity received by an inductively coupled element (ICE), such as a transformer, during an MR tracking sequence is dependent on the relative orientation of the ICE and the tracking pulse sequence projection planes. For instance, when an MR tracking plane projection is oriented orthogonally to the long axis of an ICE, then the magnetic field present at tissues surrounding the ICE structure may be substantially the same, resulting in a large projection signal from the ICE in that plane. Alternately, if the MR tracking plane projection is parallel to the long axis of the ICE, then the tissues around the ICE will have different magnetic fields applied to them by the MR gradient system, resulting in a lower more broad signal projection received by the ICE.

Similarly, an MR tracking coil may receive varying levels of MR signal from surrounding tissues depending on its physical characteristics and relative orientation to the tracking coil projections. In some orientations, the MR tracking signal received by the tracking coil may not be large enough to detect.

Therefore, in a device that utilizes an MR tracking coil with an ICE in the transmission line, the ICE location can be mistaken for the tracking coil locations, depending upon the orientation of the device, resulting in a false location of the device.

Similarly, in a device that utilizes an MR tracking coil without an ICE in the transmission line, tracking effectiveness can be variable depending upon the orientation of the device, resulting in loss of tracking.

Since catheter orientation is variable throughout a procedure, no one set of tracking projection planes is always optimal.

This invention is a system that uses varying projection planes to track medical device. In one embodiment, each tracking pulse sequence uses one or more different projection plane(s). In this way, the probability of prolonged false or poor device tracking is minimized, since the relative orientation of the device to the tracking projection plane(s) is continuously variable.

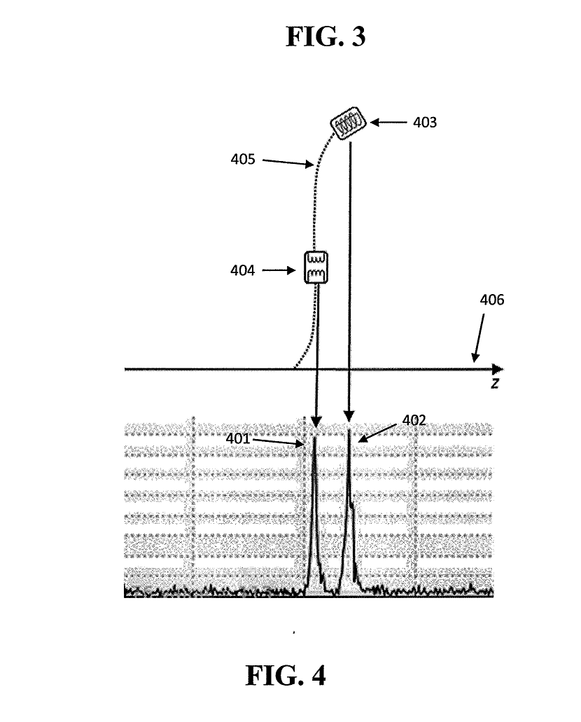

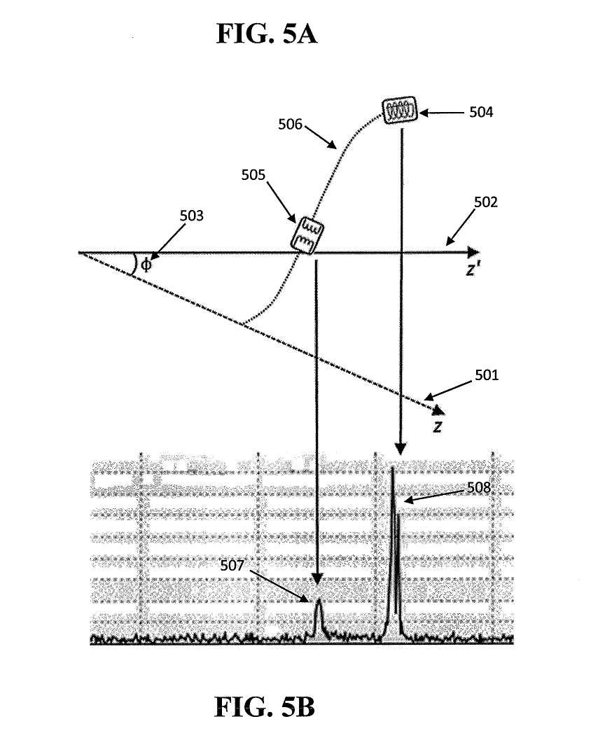

In the following example, we use only one projection plane, such that the invention can be described more easily with figures. In FIGS. 3 and 4, the ICE signal projected onto the z axis is larger than the tracking coil singles projected onto the same axis. This is because the ICE is oriented sympathetically to the magnetic field gradient, whereas the tracking coil is not. In FIG. 5, the orientations of the ICE and tracking coil, relative to the tracking projection, are such that the tracking coil signal is larger than the ICE signal, as intended.

By creating and continually using varying projection planes, the system can either collect several sets of projections before determining the location of the tracking coil, or it can determine the tracking coil's location with each projection and check for inconsistencies between projections before rendering the final tracking coil location.

A further advantage of using varying projection planes is, the signal acquired from the transformer along the transmission line can be used to depict the body of the actively tracked medical device, such as the shaft or deflection region of a catheter. This can be achieved by interpolating a line between the position of the transformer element within the transmission line and the tracking coil. A curvature can be added to the line segment and gradually increased until the arc length of the line segment is approximately equal to the predefined length. The direction of the curve can be determined by virtually connecting the transformer position to the distal most tracking coil position, then the curve of the line segment is increased towards the proximal coil position.

Using multiple different projection planes (or orthogonal coordinate systems) for a tracking acquisition also allows for the following:

Use averaging of multiple calculated tracking element positions to minimize errors associated with any single acquisition/calculation.

Use multiple projections from a single tracking coil along with an understanding of the sensitivity of the tracking coil to identify specific aspects of the tracking coil, such as the location of its edges and center.

This could also allow a single coil to provide two tracking locations (for example, the each edge of the coil could be used as a distinct tracking location.

Multiple projections could also be used with signal processing to allow individual tracking coils to be located closer to one another.

Other disclosures not directly related to the use of multiple projections include communicating with multiple tracking coils using a single transmission line and imaging using tracking coils for receive antennas to produce a region of high intensity in the MR image and using that high intensity region to identify the catheter and/or tracking coils.

Although the present invention has been described with reference to particular embodiments, those of ordinary skill in the art will recognize that changes may be made in form and detail without departing from the spirit and scope of the invention.

What is claimed is:

1. A method for projecting a broad tracking signal received by an inductively coupled element, such as a transformer during an MR tracking sequence comprising:
varying one or more projection planes of a tracking signal;
acquiring the tracking signal from a transformer element along a transmission line to depict the body of the actively tracked medical device;
interpolating a line between a position of the transformer element within the transmission line and a tracking coil;
adding a curvature to a line segment and increasing the curvature until an arc length of the line segment is approximately equal to a predefined length;
determining a direction of the curvature by virtually connecting a position of the transformer to a distal most tracking coil position; and
increasing the curvature of the line segment towards a proximal coil position.
